(19) United States
(12) Patent Application Publication
(10) Pub. No.: US 2023/0075854 A1
(43) Pub. Date: Mar. 9, 2023

(54) VIRUS-FREE CELL CULTURES

(71) Applicant: BIOMARIN PHARMACEUTICAL INC., Novato, CA (US)

(72) Inventors: Michael LOCHRIE, Hayward, CA (US); Peter COLOSI, Novato, CA (US); Barrie CARTER, Seattle, WA (US); Tomas CINEK, Novato, CA (US)

(21) Appl. No.: 17/760,227

(22) PCT Filed: Feb. 9, 2021
(86) PCT No.: PCT/US21/17301

§ 371 Date: Aug. 5, 2022

Related U.S. Application Data
(60) Provisional application No. 62/972,211, filed on Feb. 10, 2020.

(51) Int. Cl. C12N 7/00

(52) U.S. Cl. CPC C12N 7/00; C12N 2760/20064

(57) ABSTRACT

The invention is directed to methods of removing adventitious virus from cells and cell lines and virus-free cells and cell lines obtainable by such methods.

### CROSS REFERENCE TO RELATED APPLICATIONS

This application claims the benefit of U.S. Provisional Patent Application No. 62/972,211, filed 10 Feb. 2020, the contents of which are hereby incorporated by reference in their entirety.

### FIELD OF INVENTION

The invention is directed to methods of removing adventitious virus such as rhabdovirus from a cell, methods of generating a virus-free cell line, and a cell or virus-free cell line produced by these methods.

### BACKGROUND

Cell cultures are frequently used to generate gene therapy vectors or biologic products. It is important that any adventitious viruses or infectious agents in such cell substrates be recognized and removed in order that they do not contaminate the final product. In some cases, viral clearance studies can be conducted to demonstrate efficient removal of such adventitious contaminants, but it may be difficult to ensure that the adventitious agent has been removed below certain limits. It may be preferable as an added enhancement to remove such contaminating viruses prior to using the cell substrate for production.

Insect cells can harbor viruses that may be undesirable in the final gene therapy or biological product, even if they do not infect humans. Removal of the virus from the cell substrate can be achieved by growing the cells in the presence of a compound that inhibits the growth of the contaminant. For example, Sf9 cells are routinely used for commercial production of recombinant proteins, vaccines, biologics or gene delivery vehicles such as recombinant adeno-associated virus (rAAV) vectors. Sf9 cells or other insect cells may be contaminated by viruses such as rhabdovirus or other viruses (Ma et al., J. Virol., 88(12): 6576-6585, 2014).

There is a need to develop further methods to efficiently remove contaminating viruses prior to using the cells for production of gene therapy vectors or biologic products.

### SUMMARY OF INVENTION

The present invention provides for methods of removing adventitious virus from a cell and provides for a virus-free cell or cell line obtainable by such methods. For example, in such a method, cells are grown at a temperature that is higher or lower than the optimal temperature range for replication of the cells or replication of the virus, for at least two passages. Thus, the cells are grown at a suboptimal temperature outside the optimal temperature range for replication of the cells or replication of the virus. After this step(s), the cells can be returned to a temperature within the optimal temperature range or maintained at the elevated or decreased temperature. Optionally, the cells are cloned by a method, after or during the growth at the elevated or decreased temperature, to achieve a monoclonal cell population. Optionally, prior to the growing step, the cells are adapted to growth at elevated or decreased temperatures in order to increase the differential temperature sensitivity between the cell and virus. In one example, growth for 8 passages results in a 4-log reduction in virus. Further passages result in undetectable levels of virus.

In another aspect, insect cells, such as Sf9 cells, contaminated by rhabdovirus are freed of the virus by growing and passaging the cells in the presence of an antiviral agent. Clones that are free of viral infection may be obtained by a combination of growth in the presence of viral inhibitors, including a combination of two or more, three or more, or four or more different viral inhibitors, and cloning by a method to achieve single-cell cloning.

The invention provides for methods of removing or curing adventitious viruses from a cell, e.g. an insect mammalian cell, comprising growing cells in the presence of an antiviral agent, for at least 2 passages, and detecting the reduction of the adventitious virus in the culture following growth in the presence of the antiviral agent. Optionally, the cells are cloned by a method to achieve a monoclonal cell population after or during growth in the presence of an antiviral agent in order to isolate cells that have been cured of adventitious virus.

Clones obtained by any of the methods described herein can be tested to detect a reduction of the adventitious virus in the cells. Clones can also be tested to determine if they maintain desired properties present in the original parent cells such as biologics or rAAV vector production level, doubling time, viability and ability to grow in suspension. In particular, the methods of the invention may remove, eliminate, prevent or cure S. frugiperda rhabdovirus (Sf-rhabdovirus) the Sf9 cells.

For example, the methods of the invention may be carried out on insect cells. In one example, the insect cells used in the methods of the invention are Spodoptera frupperda cells, Trichophusia ni cells, Ascalapha odorata cells or Aedes albopictus cells. The insect cells used in the methods of the invention include Sf9, Se301, SeIZD2109, SeUCR1, Sf900+, Sf21, BTI-TN-5B1-4, MG-1, Tn368, HzAm1, Ha2302, Hz2E, BmN, Ao38 and High Five.

The methods of the invention may be carried out to remove any adventitious virus that infects cells. For example, the methods of the invention may be used to remove an adventitious virus selected from the group including Mononegavirales, rhabdovirus, Sf-rhabdovirus, Cytohabdovirus, Ephemeovirus, Nodaviruis, Lyssavirus, Norvihabdovirus, Nucleorhabdovirus, Perhabdovirus, Sigmavirus, Sinistar-like virus, Sprivivirus, Tibrovirus, Tupavirus, Vesiculorirus, Taastrup virus, Nakha virus, Chandipura virus, Lettuce yellow mottle virus, Ivy vein banding virus isolate, and Durham virus. In another embodiment, the invention provides for methods of removing, preventing or eliminating Nodavirus in insect cells, such as Trichoplusia ni cells, or High Five cells.

The term “cell line” refers to a population of cells capable of continuous or prolonged growth and division in vitro. Often, cell lines are clonal populations derived from a single progenitor cell. The term “cell culture” also refers to the growth of animal or plant cells in a favorable artificial environment. Cells may be removed from animal or plant tissue directly and separated by enzymatic or mechanical means before cultivation, or they may be derived from a cell line or cell strain that has already been established. The methods of the invention may be carried out with primary cells of an insect or mammalian origin, established cell lines, or subclones of either. The methods of the invention may be carried out with cells in liquid or semi-solid culture media with anchorage-dependent cells which are adhered to a solid or semi-solid substrate. In addition, any of the methods of the invention may be carried out in suspension cultures wherein the cells are floating in agitated media such as rotating media. The cells may be grown in any size culture vessel including large scale vessels and bioreactors. The term “passage” refers to the subculturing of cells by harvesting the cells from a culture vessel and re-seeding them into the same or another culture vessel. The subculturing may require disassociation of adherent cells from the culture vessel.

Removing adventitious viruses from cells refers to reducing at least some of the viral contamination in the cells. Cells with adventitious viruses removed are capable of producing rAAV vectors, lentivirus vectors, or biologic products with at least a reduced adventitious viral concentration as compared to cells contaminated with adventitious viruses and not produced by the methods of the invention. For example, removing adventitious viruses from the cells may result in a 1-10 log reduction in the amount of virus in the purified biological product.

Curing cells of adventitious virus refers to eliminating the viral contamination in cells, which results in cells capable of producing rAAV vectors, lentivirus vectors, or biologic products with a negligible level of adventitious virus. The term “elimination” or “eliminating” refers to a reduction of the viral titer or viral particles in cells and/or in cell culture supernatant to a level that is undetectable in view of the sensitivity of the assay utilized. Alternatively, cells may be considered cured when the amount of virus in the biological product or rAAV vector is equal to or less than the standard required by the FDA, if that standard is available.

Any of the methods described herein may comprise growing the cells using the methods for at least two passages, or at least three passages, or at least four passages, or at least five passages, or at least six passages, or at least seven passages, or at least eight passages, or at least nine passages, or at least 10 passages or at least 15 passages or at least 20, 25, 30, 35, 40, 45, 50, 55, 60, 65, or 70 passages or more.

The cells are optionally cloned by a method, after or during the growing step, to achieve monocolonality. The term “monoclonal” or “monocolonality” refers to being derived from a single cell. The cloning method includes different cloning methods, which can ensure for the monocolonality of the clones or attempt to achieve monocolonality of the clones such that the steps and conditions of the different cloning methods are conducive for isolating monoclonal clones. For example, the cloning method includes the step of dispensing ≤1 cell per well manually or with a cell-dispensing instrument such as a cell sorter or microfluidics-based system such as a cell printer. Since the outgrowth efficiency can tend to be low, this cloning method can further include dispensing ≤1 cell per well into a plurality of wells. After dispensing the cells, the wells are scanned manually (e.g. using a high-powered microscope lens) or with an instrument such as an imager to quantify the number of cells dispensed in the wells. In another example, the cloning efficiency of the cells is estimated prior to dispensing step. The cloning efficiency can be estimated by culturing a low number of cells (e.g. 100-10000 cells) for a time period, counting the number of colonies, and dividing the number of colonies by the number of cells originally cultured. It is noted that the cloning efficiency can depend on the starting cell density. From the cloning efficiency, one estimates the number of single cell clones (e.g. 30 clones per plate) that form from a number of cells and dispenses the number of cells onto a plate. The plate is monitored manually (e.g. using a high-powered microscope lens) or with an instrument such as an imager to identify colonies on the plate to confirm the formation of colonies and clones. From the monitoring, one can visually identify colonies containing a plurality of cells (e.g. 100 cells per colony). Other examples of the other cloning methods that can yield single-cell clones include, but are not limited to, serial limiting dilutions, cloning in a semisolid medium, cloning using an agarose overlay method, using cloning rings/cylinders, etc. Cloning by serial limiting dilution refers to a technique for attempting to obtain a monoclonal cell population starting from a polyclonal mass of cells. This is achieved by carrying out a series of increasing dilutions of the parent (polyclonal) cell culture. For example, a suspension of parent cells is diluted a number of times depending on cell number in the starting population, as well as the viability and characteristics of the cells being cloned. In the present invention, the series of increasing dilutions of cells growing according to any of the methods described herein results in a monoclonal culture of cells cured of adventitious virus. In one embodiment, a cell culture may be diluted such that only a single cell from a polyclonal parent culture is isolated and further propagated. For the different cloning methods, the cells are cultured in conditions for single-cell outgrowth. Examples of conditions for single-cell outgrowth include culture medium (e.g. conditioned media) and wells/plates capable for supporting single-cell outgrowth.

The cloning method can also include a verification analysis to improve the probability of isolating monoclonal clones. In one example, the verification analysis includes the use of imaging technology in conjunction with the cell-dispensing instrument to capture a sequence of single-cell images on the cell-dispensing instrument prior to dispensing the cells. In another example, next generation sequencing of the genome of isolated clones can be used to evaluate monoclonality.

These methods may further comprise the step of quantitatively measuring the adventitious virus prior to the methods and after the methods. The invention provides for methods in which removing the adventitious virus includes reducing the number of virus in the insect or mammalian cells, for example the reduction of the rhabdovirus in the cells and/or in the culture supernatant is statistically significant compared to an untreated control.

In any of methods of the invention, removing the adventitious virus may reduce the viral titer or viral particles by at least 1 log reduction compared to the culture before treatment, reduce the viral titer or viral particles by at least 2 log reduction compared to the culture before treatment, reduce the viral titer or viral particles by at least 3 log reduction compared to the culture before treatment, reduce the viral titer or viral particles by at least 4 log reduction compared to the culture before treatment, reduce the viral titer or viral particles by at least 5 log reduction compared to the culture before treatment, reduce the viral titer or viral particles by at least 6 log reduction compared to the culture before treatment, reduce the viral titer or viral particles by at least 7 log reduction compared to the culture before treatment, reduce the viral titer or viral particles by at least 8 log reduction compared to the culture before treatment, reduce the viral titer or viral particles by at least 9 log reduction compared to the culture before treatment, or reduce the viral titer or viral particles by at least 10 or more log reduction compared to the culture before treatment.

For example, in certain embodiments, a cell or cell line would be considered cured if the amount of viral titer or viral particles in the biologic product or rAAV vector is below a level of detection of an analytical assay such as quantitative reverse transcription polymerase chain reaction (RT-qPCR) or Sanger sequencing or if viral genetic sequences are not detected when analyzing the genome of the cells by a process such as polymerase chain reaction (PCR) or sequencing.

In addition, the invention provides for methods in which the adventitious virus is eliminated from the insect or mammalian cells. The methods of the invention also contemplate using two or more antiviral agents that each reduce the viral titer or viral particles in order to further reduce or achieve elimination of the virus from the insect cells.

In any of the methods of the invention, the adventitious virus is detected within the media or supernatant collected from the cell culture or in the cells themselves. For example, the virus is detected by or the viral titer is determined by measuring the concentration of a viral element (e.g. genome, RNA, protein, etc.) in the media or collected supernatant. The adventitious virus also may be detected by measuring viral infection and replication using methods that detect cytopathic effects, plaque formation, or viral amplification. Examples of detection methods include Median Tissue Culture Infectious Dose, TCID50, enzyme-linked immunosorbent (ELISA) assays, electron microscopy, RT-qPCR, quantitative polymerase chain reaction (qPCR) for DNA or genome detection, droplet digital PCR (ddPCR)), reverse transcription droplet digital PCR (RT-ddPCR), Sanger sequencing, or next generation sequencing.

Antiviral agent refers to any compound that acts to alter the activity or production of a viral gene product or a cellular gene product that a virus depends on for replication or infectivity. For example, the antiviral agent could inhibit or enhance the activity of the viral gene product or the cellular gene product or down regulate or upregulate the production of the viral gene product or the cellular gene product such that the replication or infectivity of the virus is inhibited. In different examples for inhibiting rhabdoviruses, compounds that alter the function or expression of the nucleocapsid (encoded by the N gene), phosphoprotein (encoded by the P gene), matrix (encoded by the M gene), envelope (encoded by the G gene), RNA-dependent RNA polymerase (encoded by the L gene), guanine methyl transferase (encoded by the L gene), messenger RNA (mRNA) capping enzyme (encoded by the L gene), or various accessory genes (for example those encoding the X and Y proteins) may be used. Additional accessory genes that may be targeted by the antiviral agent include, for example, NV in IHNV Novirhabdovirus; α1, α2, β, γ, G_{NS}, in BEFV Ephemerovirus; α1, α2, β, G_{NS}, in OBOV Ephemerovirus; α1, α2, β, γ, δ, G_{NS}, in KOTV Ephemerovirus; U1, U2, U3, U4, U5, U6, U7, G_{NS}, in NGAV Hart Park Group; U1, U2, U3, U4, U5 in WONV Hart Park Group; U1, U2, U3 in TIBV Tibrovirus; U1, U2, U3 in CPV Tibrovirus; X in DmSV Sigmavirus; C, SH in TUPV Rhabdovirus; C, SH in DURV Rhabdovirus; C, C1, C2, SH in OAKV Rhabdovirus; and SH in SCRV Rhabdovirus (see, e.g., Walker et al., Virus Res., 162:110-125 (2011)).

In any of the methods of the invention, the antiviral agent may be siRNAs designed to inhibit specific viral genes (e.g. L gene), exonucleases, endonucleases, deoxyribonucleases, ribonucleases, ribozymes, zinc finger nuclease(s) (ZFN), transcription activator-like effector nuclease(s) (TALEN), clustered regularly interspaced short palindromic repeats (CRISPR) Cas type and CRISPR-associated proteins (e.g. Cas9, Cas13, or Csfl1), phosphonoformate, phosphonoacetate, ara-ATP, ribavirin, sinefungin, selenomethionine, chloroquine, Augonaute up-regulators, Dicer up-regulators, Ars2 up-regulators Argonaute, Dicer, metformin, saquinavor, nelfinavir, cystatin A, MG132, bortezomib, leptomycin B, azodicarbonamide or sodium selenite.

In any of the methods of the invention, the antiviral agent alters the activity or production of a viral gene product or a cellular gene product such that the replication or infectivity of the virus is inhibited. For example, expression of the viral gene products encoded by the X gene, N gene, P gene, M gene, G gene or L gene may be altered by the antiviral agent.

A further aspect of the invention provides a host cell line produced by or obtainable by any of the methods described herein, that has no detectable virus, either intracellularly or extracellularly. The cells may be any of the cells described herein, e.g. insect cells or mammalian cells, and the virus may be any of the viruses described herein, e.g. rhabdovirus or nodavirus. In some embodiments, the host cell line is a Spodoptera frupperda host cell line that has no detectable rhabdovirus, e.g. by qPCR techniques. In some embodiments, the host cell line is a Trichoplusia ni host cell line, e.g. a High Five host cell line, that has no detectable nodavirus.

### DETAILED DESCRIPTION

As required, detailed embodiments of the present disclosure are disclosed herein; however, it is to be understood that the disclosed embodiments are merely exemplary and may be embodied in various and alternative forms.

Except in the examples, or where otherwise expressly indicated, all numerical quantities in this description indicating amounts of material or conditions of reaction and/or use are to be understood as modified by the word “about”. The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Unless indicated otherwise, all technical and scientific terms used herein have the same meaning as commonly understood by one of ordinary skill in the art to which the present disclosure belongs.

It is also to be understood that this disclosure is not limited to the specific embodiments and methods described below, as specific components and/or conditions may, of course, vary. Furthermore, the terminology used herein is used only for describing particular embodiments and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The terms “or” and “and” can be used interchangeably and can be understood to mean “and/or”.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

### Cell Types Used in Biologic or Vector Production

The methods may be carried out on any cell type that allows for production of rAAV or biologic products and which can be maintained in culture. The cells include invertebrate cells, e.g. insect cells, and mammalian cells.

The methods of the invention may be carried out with any invertebrate cell type which allows for production of rAAV or biologic products and which can be maintained in culture. For example, the insect cell line used can be from Spodoptera frupperda, such as SF9, SF21, SF900+, drosophila cell lines, mosquito cell lines, e.g., Aedes albopictus derived cell lines, domestic silkworm cell lines, e.g. Bombyxmori cell lines, Trichoplusia ni cell lines such as High Five cells or Lepidoptera cell lines such as Ascalapha odorata cell lines. Preferred insect cells or cell lines are cells from the insect species which are susceptible to baculovirus infection, including High Five, Sf9, Se301, SeIZD2109, SeUCR1, Sf9, Sf900+, Sf21, BTI-TN-5B1-4, MG-1, Tn368, HzAm1, BM-N, Ha2302, Hz2E5 and Ao38.

Baculoviruses are enveloped DNA viruses of arthropods, two members of which are well known expression vectors for producing recombinant proteins in cell cultures. Baculoviruses have circular double-stranded genomes (80-200 kilobase pairs (kbp)) which can be engineered to allow the delivery of large genomic content to specific cells. The viruses used as a vector are generally Autographa californica multicapsid nucleopolyhedrovirus (AcMNPV) or Bombyx mori nucleopolyhedrovirus (BmNPV) (Kato et al., 2010).

Baculoviruses are commonly used for the infection of insect cells for the expression of recombinant proteins. In particular, expression of heterologous genes in insects can be accomplished as described in for instance in U.S. Pat. No. 4,745,051; EP 127,839; EP 155,476; Vlak et al., (1988), Journal of General Virology, vol. 68, pp 765-776; Miller et al., (1988), Annual Review of Microbiology, vol. 42, pp 177-179; Carbonell et al., (1998), Gene, vol. 73, Issue 2, pp 409-418; Maeda et al., (1985), Nature, vol. 315, pp 592-594; Lebacq-Veheyden et al., (1988), Molecular and Cellular Biology, vol. 8, no. 8, pp 3129-3135; Smith et al., (1985), PNAS, vol. 82, pp 8404-8408; and Miyajima et al., (1987), Gene, vol. 58, pp 273-281. Numerous baculovirus strains and variants and corresponding permissive insect host cells that can be used for protein production are described in Luckow et al., (1988), Nature Biotechnology, vol. 6, pp 47-55; Maeda et al., (1985), Nature, vol. 315, pp 592-594; and McKenna et al., (1998), Journal of Invertebrate Pathology, vol. 71, Issue 1, pp 82-90. In another aspect of the invention, the methods of the invention are also carried out with any mammalian cell type which allows for replication of rAAV or production of biologic products, and which can be maintained in culture. Preferred mammalian cells used can be HEK293, HeLa, CHO, NS0, SP2/0, PER.C6, Vero, RD, BHK, HT 1080, A549, Cos-7, ARPE-19 and MRC-5 cells.

### Passaging at a Temperature Higher or Lower than the Optimal Temperature Range for Virus Replication

The methods described herein involve growing the cells of the cell culture at a temperature that is higher (or lower) than the optimal temperature range for virus replication, preferably viral genome replication. Without being limited by theory, the methods may take advantage of differential temperature sensitivity of virus replication or virus genome replication as compared to cell replication. Viruses replicate within cells, in part, using proteins encoded by the viral genome. For example, Sf-rhabdovirus genome replication, and hence virus production, is dependent on expression of the protein product of the Sf-rhabdovirus L gene. When the optimal temperature range for virus replication is unknown, the optimal temperature range for its typical host cell can be used as a surrogate temperature range. Thus, the methods described herein involve growing the cells of the cell culture at a temperature that is higher (or lower) than the optimal temperature range for the typical host cell of the virus.

The L protein is a multifunctional protein with viral RNA polymerase, mRNA capping, and mRNA polyadenylation activities. If the expression or enzymatic activities of the Sf-rhabdovirus L protein were reduced, production of anti-genomes and genomes would be reduced. This, in turn, would (a) prevent generation of new genomes and assembly of new infectious virions, and (b) prevent production of viral mRNAs which in turn would eliminate viral protein expression and virus production. Therefore, complete elimination of genomes from Sf9 cells would result in curing them of Sf-rhabdovirus production. In contrast, inhibiting production of the viral envelope protein, for example, might only result in the production of noninfectious virus.

Without being bound by a theory of the invention, it is hypothesized that the Sf-rhabdovirus RNA polymerase is more sensitive to higher growth temperatures than Sf9 cells because the virus RNA polymerase evolved to have optimal activity at the optimal growth temperature of Sf9 cells (27-28 degrees Centigrade (° C.). Sf9 cells as shown herein can be adapted to growth at temperatures as high as 35° C., much higher than the optimal growth temperature. For example, the SF9 cells are grown at a temperature of more than 28° C. In various embodiments, the SF9 cells are grown at 28.1° C., 28.2° C., 28.3° C., 28.4° C., 28.5° C., 28.6° C., 28.7° C., 28.8° C., 28.9° C., 29° C., 29.1° C., 29.2° C., 29.3° C., 29.4° C., 29.5° C., 29.6° C., 29.7° C., 29.8° C., 29.9° C., 30° C., 30.1° C., 30.2° C., 30.3° C., 30.4° C., 30.5° C., 30.6° C., 30.7° C., 30.8° C., 30.9° C., 31° C., 31.1° C., 31.2° C., 31.3° C., 31.4° C., 31.5° C., 31.6° C., 31.7° C., 31.8° C., 31.9° C., 32° C., 32.1° C., 32.2° C., 32.3° C., 32.4° C., 32.5° C., 32.6° C., 32.7° C., 32.8° C., 32.9° C., 33° C., 33.1° C., 33.2° C., 33.3° C., 33.4° C., 33.5° C., 33.6° C., 33.7° C., 33.8° C., 33.9° C., 34° C., 34.1° C., 34.2° C., 34.3° C., 34.4° C., 34.5° C., 34.6° C., 34.7° C., 34.8° C., 34.9° C., or 35° C. In various embodiments, the temperature at which the SF9 cells are grown is a range between any two of the above specified temperatures. The elevated temperature may range, for example, from about 29° C. to about 35° C., or about 31° C. to about 35° C., or about 32° C. to about 35° C., or about 33° C. to about 35° C.

Sf9 cells can also grow at temperatures that are less than 27° C. For example, the SF9 cells are grown at a temperature of less than 27° C. In various embodiments, the SF9 cells are grown at 20° C., 20.1° C., 20.2° C., 20.3° C., 20.4° C., 20.5° C., 20.6° C., 20.7° C., 20.8° C., 20.9° C., 21° C., 21.1° C., 21.2° C., 21.3° C., 21.4° C., 21.5° C., 21.6° C., 21.7° C., 21.8° C., 21.9° C., 22° C., 22.1° C., 22.2° C., 22.3° C., 22.4° C., 22.5° C., 22.6° C., 22.7° C., 22.8° C., 22.9° C., 23° C., 23.1° C., 23.2° C., 23.3° C., 23.4° C., 23.5° C., 23.6° C., 23.7° C., 23.8° C., 23.9° C., 24° C., 24.1° C., 24.2° C., 24.3° C., 24.4° C., 24.5° C., 24.6° C., 24.7° C., 24.8° C., 24.9° C., 25° C., 25.1° C., 25.2° C., 25.3° C., 25.4° C., 25.5° C., 25.6° C., 25.7° C., 25.8° C., 25.9° C., 26° C., 26.1° C., 26.2° C., 26.3° C., 26.4° C., 26.5° C., 26.6° C., 26.7° C., 26.8° C., or 26.9° C. In various embodiments, the temperature at which the SF9 cells are grown is a range between any two of the above specified temperatures. The reduced temperature may range, for example, from about 20° C. to about 25° C. or about 20° C. to about 24° C. or about 21° C. to about 25° C. or about 21° C. to about 24° C. Many different types of cells have multiple mechanisms to survive some degree of growth at elevated temperatures, so the methods herein are contemplated to be applicable to other types of cells.

It is also noted that mammalian cells such as HEK293, HeLa, CHO, NS0, SP2/0, PER.C6, Vero, RD, BHK, HT 1080, A549, Cos-7, ARPE-19 and MRC-5 cells have optimal activity at an optimal growth temperature of 37° C. or an optimal growth temperature range of 35° C. to 38° C. In various embodiments, the mammalian cells are grown at a temperature that is higher or lower than the optimal growth temperature (e.g. 37° C.) or the optimal growth temperature range (e.g. 35° C. to 38° C.). For example, the mammalian cells are grown at a temperature ranging from 25° C. to less than 37° C. (e.g. 25° C., 26° C., 27° C., 28° C., 29° C., 30° C., 31° C., 32° C., 33° C., 34° C., 35° C., 36° C.) or a temperature ranging from greater than 37° C. to 44° C. (e.g. 38° C., 39° C., 40° C., 41° C., 42° C., 43° C., or 44° C.). In other examples, the mammalian cells are grown at a temperature ranging from 25° C. to less than 35° C. (e.g. 25° C., 26° C., 27° C., 28° C., 29° C., 30° C., 31° C., 32° C., 33° C., 34° C.) or a temperature ranging from greater than 38° C. to 44° C. (e.g. 39° C., 40° C., 41° C., 42° C., 43° C., or 44° C.).

If the rate at which viral genomes replicate is less than the rate of cell division, then eventually the viral genomes are diluted in number and cells that are cured of Sf-rhabdovirus production will result. If residual genomes or mRNAs remain in the culture, a cell line without any residual genomes or mRNAs can be derived from the population by single-cell cloning if cells without any Sf-rhabdovirus RNA exist at a reasonable frequency.

The data herein shows that 8 passages at elevated temperature results in cells with a 4-log fold decrease in virus. Further passages result in a viral load that is undetectable by assay.

According to this aspect of the invention, in cases where the methods are conducted at an elevated temperature, the temperature may be increased gradually by, e.g. 1° C. after each passage, or 1° C. after 2 passages at the same temperature, or 1° C. after 3 passages at the same temperature. In various embodiments, the temperature after each passage is increased by 0.1° C., 0.2° C., 0.3° C., 0.4° C., 0.5° C., 0.6° C., 0.7° C., 0.8° C., 0.9° C., 1° C., 1.1° C., 1.2° C., 1.3° C., 1.4° C., 1.5° C., 1.6° C., 1.7° C., 1.8° C., 1.9° C., 2° C., 2.1° C., 2.2° C., 2.3° C., 2.4° C., 2.5° C., 2.6° C., 2.7° C., 2.8° C., 2.9° C., or 3° C. In various embodiments, the temperature increase after each passage is a range between any two of the above specified temperatures. For example, the temperature increase after each passage ranges from 0.1° C. to 3° C., 0.2° C. to 2.9° C., 0.3° C. to 2.8° C., 0.4° C. to 2.7° C., 0.5° C. to 2.6° C., 0.6° C. to 2.5° C., 0.7° C. to 2.4° C., 0.8° C. to 2.3° C., 0.9° C. to 2.2° C., 1° C. to 2.1° C., or 1.1° C. to 2° C. In cases where the methods are conducted at a decreased temperature, the temperature may be decreased by, e.g. 1° C. after each passage, or 1° C. after 2 passages at the same temperature, or 1° C. after 3 passages at the same temperature. In various embodiments, the temperature after each passage is decreased by 0.1° C., 0.2° C., 0.3° C., 0.4° C., 0.5° C., 0.6° C., 0.7° C., 0.8° C., 0.9° C., 1° C., 1.1° C., 1.2° C., 1.3° C., 1.4° C., 1.5° C., 1.6° C., 1.7° C., 1.8° C., 1.9° C., 2° C., 2.1° C., 2.2° C., 2.3° C., 2.4° C., 2.5° C., 2.6° C., 2.7° C., 2.8° C., 2.9° C., or 3° C. In various embodiments, the temperature decrease after each passage is a range between any two of the above specified temperatures. For example, the temperature decrease after each passage ranges from 0.1° C. to 3° C., 0.2° C. to 2.9° C., 0.3° C. to 2.8° C., 0.4° C. to 2.7° C., 0.5° C. to 2.6° C., 0.6° C. to 2.5° C., 0.7° C. to 2.4° C., 0.8° C. to 2.3° C., 0.9° C. to 2.2° C., 1° C. to 2.1° C., or 1.1° C. to 2° C. The speed of the temperature change depends on how well the cells are growing. If the cells are growing well, the temperature can be changed by a degree in the next passage. If the cells are growing more slowly, they may be maintained at the same temperature for two or three or more passages until they adapt, after which the temperature can be changed by a degree in the next passage. The temperature is continually increased or decreased until the desired end temperature is reached.

The end temperature may be 1° C., 2° C., 3° C., 4° C., 5° C., 6° C., 7° C., 8° C. or more degrees Centigrade higher (or lower) than either the optimal temperature range for replication of the cells, e.g. the typical host cells for the virus, or the optimal temperature range for replication of the virus. In various embodiments, the end temperature is a range between any two of the above specified temperatures. For example, the end temperature may be about 2° C. to about 7° C. higher, or about 3° C. to about 7° C. higher, or about 4° C. to about 7° C. higher, or about 2° C. to about 6° C. higher, or about 3° C. to about 6° C. higher, or about 4° C. to about 6° C. higher than the upper end of the optimal temperature range for replication of the virus. As another example, the end temperature may be about 2° C. to about 7° C. lower, or about 3° C. to about 7° C. lower, or about 4° C. to about 7° C. lower, or about 2° C. to about 6° C. lower, or about 3° C. to about 6° C. lower, or about 4° C. to about 6° C. lower than the lower end of the optimal temperature range for replication of the virus.

After these steps (culturing the cells for a period of time at the elevated or reduced temperature), the cells can (a) be returned to a temperature at the optimal temperature range for replication of the cells, or (b) be maintained at the elevated (or decreased) temperature. For example, the optimal temperature range for replication of SF9 cells is 27° C. to 28° C., and the SF9 cells may be returned to that optimal temperature range after being grown for a period of time at an elevated temperature.

### Antiviral Agents

Antiviral agent refers to any compound that inhibits either the activity or production of a viral gene product or a cellular gene product that a virus depends on for replication. For example, in the case of rhabdoviruses, compounds that inhibit the function of the nucleocapsid (encoded by the N gene), phosphoprotein (encoded by the P gene), matrix (encoded by the M gene), envelope (encoded by the G gene), RNA-dependent RNA polymerase (encoded by the L gene), or the X and Y proteins may be used in any of the methods of the invention. In addition, agents which inhibit expression of proteins encoded by the N gene, P gene, M gene, G gene, or L gene or agents that inhibit the expression of the X and Y proteins of rhabdovirus, such as antisense oligonucleotides, siRNA, dsRNA, ZFN, TALEN, CRISPR, or other gene editing nuclease/guide RNA system may be used in any of the methods of the invention.

The methods of the invention may be carried out with one or more antiviral agents. The invention contemplates using any combination of the antiviral agents that removes or cures viral contamination in the cells. The antiviral agents exhibit a low level of cytotoxicity to the cells or are not cytotoxic to cells. The cytotoxicity can be monitored using different methods, where different concentrations of antivirals are assessed. For example, cells in culture (e.g. multi-well plate) are assessed manually with a microscope or using a cell viability dye (e.g. 4′,6-diamidino-2-phenylindole, ethidium bromide, propidium iodide, 7-AAD, LIVE/DEAD® Stain, etc.) or other indicator(s) that allows for high throughput analysis of cell viability. In other examples, the methods of the invention may be carried out with a combination of two or more antiviral agents having a synergistic or additive effect on reducing or eliminating viral titer or viral particles. For example, the methods of the invention are carried out with two or more antiviral agents in which the antiviral agents act at different phases of the viral life cycle or are directed at different targets. Alternatively, the methods of the invention are carried out with two or more antiviral agents in which the antiviral agents are directed to the same target but inhibit viral activity by different mechanisms.

For example, any of the methods of the invention may use broad-specificity inhibitors of viral proteins, such as RNA-dependent RNA polymerases which are required for genome replication. These broad-specificity inhibitors include phosphonoformate, phosphonoacetate, ara-ATP, and ribavirin.

Any of the methods of the invention may use inhibitors of viral proteins such as inhibitors of methyl transferases and inhibitors of envelope proteins. Methyl transferases are required for proper 5′ cap synthesis, and examples of inhibitor of methyl transferase include sinefungin and S-adenosyl methionine analogs such as selenomethionine. Envelope proteins are required for infection of cells. Examples of inhibitors of envelope proteins include compounds such as chloroquine that prevent endosomes from attaining the low pH required for the envelope proteins to mediate capsid escape from the endosome. In addition, many envelope proteins are disulphide bonded and glycosylated so disulphide bond reducing agents (e.g., dithiothreitol) and glycosylation inhibitors (e.g., tunicamycin) may also interfere with their function.

Furthermore, neutralizing antibodies that bind to and inhibit the viral envelope can be isolated by standard methods (e.g., immunization with peptides or recombinant fragments of envelope, or against concentrated virus; phage display selection of recombinant antibodies) and used to inhibit infection of cells by virus that escapes inhibition by other methods and is produced by the cell. Use of neutralizing antibodies to reduce or eliminate viral infection is a classic method but there is a risk that in long term culture viral mutants, which are resistant to neutralization, may be selected rendering such antibodies ineffective.

Inhibition of RNA virus infection in insect cells is known to involve components of the RNAi pathway such as Argonaute and Dicer. Therefore, compounds that increase the activity or synthesis of those proteins could have an antiviral effect and may be used in any of the methods of the invention. For example, Ars2 which interacts with Dicer and confers arsenite resistance, might be upregulated by arsenite. Metformin has been reported to upregulate dicer expression. In particular, the methods of the invention may be carried out with arsenite, compounds that upregulate arsenite expression or activity or metformin.

Inhibitors of cellular genes required for viral replication may be used in any of the methods of the invention. For example, these inhibitors include aspartyl proteases such as saquinavor, nelfinavir or cystatin A. Inhibitors of cellular proteins required for viral replication may be used in any of the methods of the invention. For example, these inhibitors include proteasomes inhibitors such as MG132 or bortezomib, and inhibitors of nuclear export proteins, such as leptomycin B.

Both viral and cellular zinc finger proteins may be involved in viral replication and inhibitors of these zinc finger proteins may be used in any of the methods of the invention. For example, compounds that specifically chelate zinc, such as azodicarbonamide or sodium selenite, will inhibit zinc fingers and may be used in any of the methods of the invention.

The invention contemplates using various combinations of the above compounds in any of the methods of the invention. For example, the invention particularly contemplates using a combination of antiviral agents that act on different pathways and by different mechanisms of action, and therefore may provide a synergistic effect that may be more potent in eliminating the target virus from the cells of interest. The effectiveness of any treatment will be a balance between efficiency of viral inhibition and lack of cytotoxicity.

### Inhibition of Gene Expression

The invention provides for methods of reducing adventitious virus in a cell culture using an antiviral agent that inhibits expression of proteins required for replication of the virus, such as proteins encoded by the N gene, P gene, M gene, G gene, or L gene. In addition, the antiviral agent can inhibit the expression of the X and Y proteins of rhabdovirus. Inhibition of gene expression is carried out with antisense oligonucleotides, siRNA, dsRNA, aptamers, or CAS nuclease/CRISPR guide RNAs. The methods of the invention may be carried out with one or more antiviral agents that inhibit gene expression or a combination of antiviral agents that inhibit gene expression and inhibit protein activity. The methods may further include step(s) of growing the cells at a temperature that is higher (or lower) than the optimal temperature range for replication of the cells or replication of the virus.

### Antisense

One method of inhibiting expression of gene products is by administering antisense oligonucleotides targeted to genes that encode proteins involved in viral replication. Antisense oligonucleotides generally are nucleic acid molecules which are complementary to and which hybridize to expression control sequences (e.g. triple helix formation) or to antiviral target protein mRNA. The antisense oligonucleotides can be synthesized by standard methods known in the art as further discussed below, e.g., by use of an automated DNA synthesizer, such as are commercially available from, for example, Biosearch, Applied Biosystems, Inc.

Antisense DNA or RNA oligonucleotides, which have a sequence that is complementary to at least a portion of the selected target gene(s) can be introduced into the cell. Typically, each antisense oligonucleotide will be complementary to the start site (5′ end) of each selected target gene. When the antisense molecule then hybridizes to the corresponding target mRNA, translation of this mRNA is prevented or reduced. The degree of complementarity between the target and antisense oligonucleotide is sufficient to form a stable duplex. The region of complementarity of the antisense oligonucleotides with the target RNA sequence may be as short as 8-11 bases, but is preferably 12-15 bases or more, e.g. 12-20 bases, or 12-25 bases. An antisense oligonucleotide of about 14-15 bases is generally long enough to have a unique complementary sequence in the viral genome. In addition, a minimum length of complementary bases may be required to achieve the requisite target binding (characterized by melting temperature, Tm), as discussed below. The antisense oligonucleotides may include a small number of mismatches, as long as the sequence is sufficiently complementary to allow hybridization with the target, and forms with the virus positive-strand or minus-strand, a heteroduplex having a Tm of 45° C. or greater. Oligonucleotides as long as 40 bases may be suitable, where at least the minimum number of bases, e.g., 8-11, preferably 12-15 bases, are complementary to the target sequence. In general, however, facilitated or active uptake in cells is optimized at oligonucleotide lengths less than about 30, preferably less than 25, and more preferably 20 or fewer bases.

The oligonucleotide may be 100% complementary to the viral nucleic acid target sequence, or it may include mismatches, e.g., to accommodate variants, as long as a heteroduplex formed between the oligonucleotide and viral nucleic acid target sequence is sufficiently stable to withstand the action of cellular nucleases and other modes of degradation which may occur in vivo. Oligonucleotide backbones which are less susceptible to cleavage by nucleases are discussed below. Mismatches, if present, are less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability. Although such an antisense oligonucleotide is not necessarily 100% complementary to the viral nucleic acid target sequence, it is effective to stably and specifically bind to the target sequence, such that a biological activity of the nucleic acid target, e.g., expression of viral protein(s), is modulated.

The stability of the duplex formed between the oligonucleotide and the target sequence is a function of the binding Tm and the susceptibility of the duplex to cellular enzymatic cleavage. The Tm of an antisense compound with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligonucleotide hybridization techniques, Methods Enzymol. Vol. 154 pp. 94-107. Each antisense oligonucleotide should have a binding Tm, with respect to a complementary-sequence RNA, of greater than body temperature and preferably greater than 50° C. Tm in the range 60-80° C. or greater are preferred. According to well-known principles, the Tm of an oligonucleotide compound, with respect to a complementary-based RNA hybrid, can be increased by increasing the ratio of C:G paired bases in the duplex, and/or by increasing the length (in base pairs) of the heteroduplex. At the same time, for purposes of optimizing cellular uptake, it may be advantageous to limit the size of the oligonucleotide. For this reason, compounds that show high Tm (50° C. or greater) at a length of 20 bases or less are generally preferred over those requiring greater than 20 bases for high Tm values.

The antisense compound may be taken up by host cells by facilitated or active transport across the host cell membrane if administered in free (non-complexed) form, or by an endocytic mechanism if administered in complexed form.

In the case where the antiviral agent is administered in free form, the antisense composition should be substantially uncharged, meaning that a majority of its inter-subunit linkages are uncharged at physiological (pH). A small number of net charges, e.g., 1-2 for a 15- to 20-mer oligonucleotide, can in fact enhance cellular uptake of certain oligonucleotides with substantially uncharged backbones. The charges may be carried on the oligonucleotide itself, e.g., in the backbone linkages, or may be terminal charged-group appendages. Preferably, the number of charged linkages is no more than one charged linkage per four uncharged linkages. More preferably, the number is no more than one charged linkage per ten, or no more than one per twenty, uncharged linkages. In one embodiment, the oligonucleotide is fully uncharged.

The antisense oligonucleotide may also be administered in complexed form, where the complexing agent is typically a polymer, e.g., a cationic lipid, polypeptide, or non-biological cationic polymer, having an opposite charge to any net charge on the antisense compound. Methods of forming complexes, including bilayer complexes, between anionic oligonucleotides and cationic lipid or other polymer components, are well known. For example, the liposomal composition LIPOFECTIN, containing the cationic lipid DOTMA (N-[1-(2,3-dioleyloxy)propyl]-N,N,N-trimethylammonium chloride) and the neutral phospholipid DOPE (dioleyl phosphatidyl ethanolamine), is widely used. After administration, the complex is taken up by cells through an endocytic mechanism, typically involving particle encapsulation in endosomal bodies.

In some instances, liposomes may be employed to facilitate uptake of the antisense oligonucleotide into cells. (See, e.g., Williams, S. A., Leukemia 10(12): 1980-1989, 1996; Lappalainen et al., Antiviral Res. 23:119, 1994; Uhlmann et al., “Antisense oligonucleotides: A new therapeutic principle,” Chemical Reviews, Volume 90, No. 4, pages 544-584, 1990; Gregoriadis, G., Chapter 14, Liposomes, Drug Carriers in Biology and Medicine, pp. 287-341, Academic Press, 1979). Hydrogels may also be used as vehicles for antisense oligonucleotide administration, for example, as described in WO 93/01286. Alternatively, the oligonucleotides may be administered in microspheres or microparticles. (See, e.g., Wu, G. Y. and Wu, C. H., J. Biol. Chem. 262:4429-4432, 1987). Alternatively, the use of gas-filled microbubbles complexed with the antisense oligonucleotides can enhance delivery to target tissues, as described in U.S. Pat. No. 6,245,747. Antisense RNA oligonucleotides may be delivered using recombinant virus that infect the target cells, e.g. baculoviruses that infect cells.

The methods of the invention may be carried out with one or more sets of antisense oligonucleotides or a combination of antisense oligonucleotides with other antiviral agents. The invention contemplates using any combination of the antisense agents that will reduce or eliminate viral titer or viral particles that is not cytotoxic. For example, the combination of two or more sets of antisense oligonucleotides may have a synergistic or additive effect on reducing or eliminating viral titer or viral particles. For example, the methods of the invention are carried out with two or more sets of antisense oligonucleotides in which the antisense oligonucleotides inhibit different targets that act at different phases of the viral life cycle. Alternatively, the methods of the invention are carried out with a set of antisense oligonucleotides and a different antiviral agent, wherein the antisense oligonucleotides and the other antiviral agent are directed to the same target but inhibit viral activity by different mechanisms. For example, RNaseH-like nucleases can be used to degrade an RNA based viral genome when a DNA oligonucleotide binds to the RNA based viral genome to form an RNA/DNA duplex.

### siRNA and dsRNA

RNA interference (RNAi) is a naturally occurring regulatory mechanism that uses small double stranded RNA (dsRNA) molecules to direct homology-dependent gene silencing. The term siRNA refers to a small interfering RNA which is capable of mediating RNA interference or gene silencing. siRNA is also known as silencing RNA.

Double-stranded RNA molecules (dsRNA) have been shown to block gene expression in a highly conserved regulatory mechanism known as RNA interference (RNAi). WO 99/32619 (Fire et al.) discloses the use of a dsRNA of at least 25 nucleotides in length to inhibit the expression of genes in C. elegans. Any of the methods of the invention may be carried out with dsRNA that inhibits expression of a gene that encodes a protein required for viral replication. The dsRNA includes an antisense strand having a region of complementarity which is complementary to at least a part of an mRNA formed in the expression of a target gene, and where the region of complementarity is less than 30 nucleotides in length, generally 19-24 nucleotides in length, and where said dsRNA, upon contact with a cell expressing the target gene, inhibits the expression of said target gene by at least 30% as assayed by, for example, a polymerase chain reaction (PCR) or branched DNA (bDNA)-based method, or by a protein-based method, such as by Western blot. The dsRNA used in the methods of the invention can further include one or more single-stranded nucleotide overhangs.

The dsRNA can be synthesized by standard methods known in the art as further discussed below, e.g., by use of an automated DNA synthesizer, such as are commercially available from, for example, Biosearch, Applied Biosystems, Inc. The dsRNA includes two RNA strands that are sufficiently complementary to hybridize to form a duplex structure. One strand of the dsRNA (the antisense strand) includes a region of complementarity that is substantially complementary, and generally fully complementary, to a target sequence, the other strand (the sense strand) includes a region that is complementary to the antisense strand, such that the two strands hybridize and form a duplex structure when combined under suitable conditions. Generally, the duplex structure is between 15 and 30 or between 25 and 30, or between 18 and 25, or between 19 and 24, or between 19 and 21, or 19, 20, or 21 base pairs in length. In one embodiment, the duplex is 19 base pairs in length. In another embodiment, the duplex is 21 base pairs in length. When two different siRNAs are used in combination, the duplex lengths can be identical or can differ.

The dsRNA comprises two RNA strands that are sufficiently complementary to hybridize to form a duplex structure. One strand of the dsRNA (the antisense strand) comprises a region of complementarity that is substantially complementary, and generally fully complementary, to a target sequence, derived from the sequence of an mRNA formed during the expression of a replication related viral gene, the other strand (the sense strand) comprises a region which is complementary to the antisense strand, such that the two strands hybridize and form a duplex structure when combined under suitable conditions. Generally, the duplex structure is between 15 and 30, more generally between 18 and 25, yet more generally between 19 and 24, and most generally between 19 and 21 base pairs in length. Similarly, the region of complementarity to the target sequence is between 15 and 30, more generally between 18 and 25, yet more generally between 19 and 24, and most generally between 19 and 21 nucleotides in length. The dsRNA of the invention may further comprise one or more single-stranded nucleotide overhang(s).

The dsRNA can be synthesized by standard methods known in the art as further discussed below, e.g., by use of an automated DNA synthesizer, such as are commercially available from, for example, Biosearch, Applied Biosystems, Inc.

The skilled person is well aware that dsRNAs comprising a duplex structure of between 20 and 23, but specifically 21, base pairs have been hailed as particularly effective in inducing RNA interference (Elbashir et al., EMBO 2001, 20:6877-6888). However, others have found that shorter or longer dsRNAs can be effective as well.

The dsRNA of the invention can contain one or more mismatches to the target sequence. In one embodiment, the dsRNA of the invention contains no more than 3 mismatches. If the antisense strand of the dsRNA contains mismatches to a target sequence, it is preferable that the area of mismatch not be located in the center of the region of complementarity. If the antisense strand of the dsRNA contains mismatches to the target sequence, it is preferable that the mismatch be restricted to 5 nucleotides from either end, for example 5, 4, 3, 2, or 1 nucleotide from either the 5′ or 3′ end of the region of complementarity.

In one embodiment, at least one end of the dsRNA has a single-stranded nucleotide overhang of 1 to 4, generally 1 or 2 nucleotides. dsRNAs having at least one nucleotide overhang have unexpectedly superior inhibitory properties than their blunt-ended counterparts. Moreover, the present inventors have discovered that the presence of only one nucleotide overhang strengthens the interference activity of the dsRNA, without affecting its overall stability. dsRNA having only one overhang has proven particularly stable and effective in vivo, as well as in a variety of cells, cell culture media, blood, and serum. Generally, the single-stranded overhang is located at the 3′-terminal end of the antisense strand or, alternatively, at the 3′-terminal end of the sense strand. The dsRNA may also have a blunt end, generally located at the 5′-end of the antisense strand. Such dsRNAs have improved stability and inhibitory activity. Generally, the antisense strand of the dsRNA has a nucleotide overhang at the 3′-end, and the 5′-end is blunt. In another embodiment, one or more of the nucleotides in the overhang is replaced with a nucleoside thiophosphate.

The dsRNA is chemically modified to enhance stability. The nucleic acids of the invention may be synthesized and/or modified by methods well established in the art, such as those described in “Current protocols in nucleic acid chemistry”, Beaucage, S. L. et al. (Edrs.), John Wiley & Sons, Inc., New York, N.Y., USA, which is hereby incorporated herein by reference. Specific examples of dsRNA compounds useful in this invention include dsRNAs containing modified backbones or no natural inter-nucleoside linkages. As defined in this specification, dsRNAs having modified backbones include those that retain a phosphorus atom in the backbone and those that do not have a phosphorus atom in the backbone.

Preferred modified dsRNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

The dsRNA may be administered using liposomes, hydrogels, microspheres, microparticles or gas-filled microbubbles as discussed above for antisense oligonucleotides in microspheres or microparticles. Antisense dsRNA oligonucleotides may be delivered using recombinant virus that infect the target cells, e.g. baculoviruses that infect cells.

The methods of the invention may be carried out with one or more siRNA or dsRNA or a combination of siRNA or dsRNA with other antiviral agents. The invention contemplates using any combination of the siRNA or dsRNA that removes or cures viral contamination in the cells. The combination(s) of siRNA or dsRNA exhibit a low level of cytotoxicity to the cells or are not cytotoxic to cells. For example, the combination of two or more siRNA or dsRNA may have synergistic or additive effect on reducing or eliminating viral titer or viral particles. For example, the methods of the invention are carried out with two or more siRNA dsRNA in which the siRNA or dsRNAs inhibit different targets that act at different phases of the viral life cycle. Alternatively, the methods of the invention are carried out with a siRNA or dsRNA and a different antiviral agent, wherein the siRNA or dsRNA and the other antiviral agent re directed to the same target but inhibit viral activity by different mechanisms.

### miRNA inhibitors

“miRNA Inhibitor” is an agent that blocks miRNA function, expression and/or processing. It is known that viruses with large genomes encode miRNAs or miRNA targets in their genome which potentially could be targets for inhibition. For instance, these molecules include but are not limited to microRNA specific antisense, microRNA sponges, tough decoy RNAs (TuD RNAs) and microRNA oligonucleotides (double-stranded, hairpin, short oligonucleotides) that inhibit miRNA interaction with a Drosha complex. MicroRNA sponges specifically inhibit miRNAs through a complementary heptameric seed sequence (Ebert, M. S, Nature Methods, Epub Aug. 12, 2007). Other methods for silencing miRNA function (derepression of miRNA targets) in the viral genome will be apparent to one of ordinary skill in the art.

The methods of the invention may be carried out with one or more miRNA(s) or a combination of miRNAs used for inhibition with other antiviral agents. The invention contemplates using any combination of the miRNA that will reduce or eliminate viral titer or viral particles that is not cytotoxic. For example, the combination of two or more miRNA may have synergistic or additive effect on reducing or eliminating viral titer or viral particles. For example, the methods of the invention are carried out with two or more miRNA in which the miRNA inhibit different targets that act at different phases of the viral life cycle. Alternatively, the methods of the invention are carried out with a miRNA and a different antiviral agent, wherein the miRNA and the other antiviral agent are directed to the same target but inhibit viral activity by different mechanisms.

### Ribozymes

“Ribozyme(s)” is an RNA molecule capable of catalyzing cleavage activity, which can be engineered to specifically target a given RNA molecule. Examples of ribozymes include hammerhead, hairpin and hepatitis delta virus ribozymes.

The methods of the invention may be carried out with one or more ribozymes used for inhibition with other antiviral agents. The invention contemplates using any combination of ribozymes that will reduce or eliminate viral titer or viral particles that is not cytotoxic. For example, the combination of two or more ribozymes may have synergistic or additive effect on reducing or eliminating viral titer or viral particles. For example, the methods of the invention are carried out with two or more ribozymes in which the ribozymes inhibit different targets that act at different phases of the viral life cycle. Alternatively, the methods of the invention are carried out with a ribozyme and a different antiviral agent, wherein the ribozyme and the other antiviral agent are directed to the same target but inhibit viral activity by different mechanisms.

### CRISPR systems

The CRISPR protein (e.g. Cas9, Cas13, Cpf1, etc.) complexed with a guide RNA is capable of modifying (e.g. cleaving, nicking, methylating, or demethylating) a target nucleic acid or a polypeptide associated with the target nucleic acid. The guide RNA provides target specificity to the complex by having a nucleotide sequence that is complementary to a sequence of a target nucleic acid.

The methods of the invention may be carried out with a CRISPR protein and guide RNA targeting an adventitious agent related polynucleotide. In targeting the adventitious agent, the CRISPR system can inhibit reproduction or the infectivity of the adventitious agent. The CRISPR system can also be used in combination with other antiviral agents. The invention also contemplates using different guide RNAs that target different adventitious agent related polynucleotides.

### Cell Culture and Assays for Detection of Virus in the Cell Culture

As previously stated, an aspect of the invention provides (a) cell line(s) produced by or obtainable by any of the methods described herein, that has no detectable virus, either intracellularly or extracellularly. Cultures of the cell or cell line(s) provided herein may optionally be at least 1 Liter(s) (L), 2 L, 3 L, 4 L or 5 L in volume. For example, preliminary pilot studies are done at 1 L, 2 L, 3 L, 4 L, 5 L, 6 L, 7 L, 8 L, 9 L, or 10 L scale. In other examples, larger cultures of the cells or cell line(s)are done at or at least at 100 L, 200 L, 300 L, 400 L, 500 L, 600 L, 700 L, 800 L, 900 L, 1000 L, 1100 L, 1200 L, 1300 L, 1400 L, 1500 L, 1600 L, 1700 L, 1800 L, 1900 L, or 2000 L scale.

The removal or curing the viral contamination from the cells or cell line(s) in culture may be monitored with assays that detect the viral genome (e.g., reverse transcriptase-quantitative polymerase chain reaction), viral particles (e.g., ELISA assays or electron microscopy), or viral replication (cytopathic effects, viral amplification, or plaque formation on uninfected cells lines), next generation (“deep”) sequencing of cellular RNAs (transcriptome profiling). Sandwich ELISA assay using antibodies that detect the external viral glycoproteins, such as the glycoproteins encoded by the Sf-rhabdovirus G gene, may be used to detect intact virus. Rhabdoviruses have a distinct “bullet-shaped” morphology that can be distinguished from other viruses by electron microscopy (as described in Ma et al. Journal of Virology 88:12, 6476-6585 (2014)). For example, a portion of the cell culture (e.g. 0.1-10 milliliter(s) (ml) or 1 ml for preliminary pilot studies or 10-100 ml for larger scale production) is sampled for the detection assay(s) such that virus could be detected with the assay(s).

### Adventitious Viruses

Insect cells and mammalian cells are commonly used for the manufacture of biological products such as biologic therapeutics, recombinant proteins, antibodies, and gene therapy vectors such as rAAV. These cells are susceptible to infection by adventitious viruses. The present invention provides methods of removing, reducing, or eliminating the adventitious viruses from such cells.

Examples of virus families known to infect cells in cell cultures include Paramyxovirus, Parvovirus, Adenovirus, Rhabdovirus, Coronavirus, Herpesvirus, Papillomavirus, and Retrovirus.

Examples of rhabdoviruses include the Sf-rhabdovirus, Cytohabdovirus such as the Lettuce Necrotic Yellow Virus, Ephemeovirus, Lyssavirus, Norvihabdovirus, Nucleorhabdovirus, Perhabdovirus, Sigmavirus, Sinistar-like virus, Sprivivirus, Tibrovirus, Tupavirus, Vesiculorirus, Taastrup virus, Nakha virus, Chandipura virus, Lettuce yellow mottle virus, Ivy vein banding virus isolate, and Durham virus. These viruses may infect invertebrate cells or mammalian cells.

For example, Spodoptera frupperda cells, such as Sf9 and Sf21 cells, are known to be susceptible to infection by viruses of the Mononegavirales order, in particular those viruses in the family rhabdovirus. In particular, Sf9 cells are known to be infected by the Sf-rhabdovirus. Trichopluisa ni cells, such as High Five cells, are known to be susceptible to infection by the Nodavirus. An example of a Nodavirus that infects High Five cells is the Flock House virus isolate TNCL (Li et al., Journal of Virology 81:20, 10890-10896 (2007); GenBank: EF690537.1).

Examples of viruses known to infect mammalian cells in cell cultures include Lyssavirus, Enterovirus, Poliovirus, Cytomegalovirus (CMV), Influenza Virus, Rhinovirus, Bunyavirus, Orthmomyxovirus, Coxsakie B, SV40, EMCV, Theiler virus, Arenavirus, Poxvirus, Murine Minute Virus (MVM), Rubella, Vaccina Virus, Epstein Bar Virus (EBV), Lymphocytic Choriomeningiitis Virus (LCMV), EDIM, LDH, Swine Vesticular Disease Virus, Reovirus, Bluetongue Virus, Hepatitis B Virus, and Hepatitits C Virus.

Other aspects and advantages of the present invention will be understood upon consideration of the following illustrative example.

### EXAMPLES

### Example 1

### Curing Sf9 Insect Cells of Sf-Rhabdovirus Infection by Culturing at Elevated Temperature

Protocol #1:

Sf9 cells are cured of adventitious virus by culturing the cells at elevated temperature. Sf9 cells were grown in SF 900 III SFM culture medium (Life technologies/Invitrogen), at a cell density on the order of 10⁶ cells/ml, in a 250 ml Erlenmeyer flask shaken at 135 rpm in 90% humidity, for approximately 3-4 days per passage. Cells began their first passage at a temperature of 28° C. In the next passage, the cells were grown at 29° C. and grew well. The cells were then adapted to higher growth temperatures by increasing the growth temperature by 1° C. increments after every passage. The cells continued to grow well until 35° C., at which temperature the growth of the cells slowed. In order to determine the number of Sf-rhabdovirus viral RNAs in cell culture media RNA was isolated, reverse transcribed into cDNA, then subjected to a qPCR to detect viral RNA. The results showed that the original cells had 2.2×10⁷ RNA/ml of media, while the cells adapted to grow at 35° C. had 2.4×10³ RNA/ml of media, a 4-log reduction in extracellular Sf-rhabdovirus production from Sf9 cells.

Rhabdovirus assay: Supernatant from cultured cells was purified to collect viral RNA using the QIAamp viral RNA mini kit (Qiagen), then reverse transcription followed by qPCR (RT-qPCR) of the resulting RNA was performed. Cell pellets were also tested using the same RT-qPCR method with a substitution of the QIAgen RNeasy mini kit (Qiagen) combined with an additional DNase digestion (Qiagen) to extract total RNA directly from the cells. An alternative kit (Zymo ZR Viral RNA Kit) was also used to extract RNA from both supernatant and cell pellets.

Protocol #2

Sf9 cells were adapted as in protocol #1 to higher growth temperatures by increasing the growth temperature by 1° C. increments after every passage until a temperature of 33° C. was reached, at which time the cells were maintained at 33° C. for 3 passages, then maintained at 34° C. for 4 passages. The cells grew well at 33° C. but grew more slowly at 34° C. The cells were then returned to their optimal temperature of 28° C. and maintained for multiple passages. After 61 passages, the amount of rhabdovirus viral DNA in the cell culture was undetectable (assay detection limit of 1×10² RNA/ml), while the original cells had 5×10⁹ RNA/ml of media. This protocol thus resulted in a greater than 5×10⁷ reduction in viral load.

Protocol #3

The cells that had been adapted to grow at 34° C., and then returned to 28° C. for multiple passages, were subjected to a second round of heat adaptation, at temperatures ranging from 35° C. to 37° C. The second round of heat adaptation further lowered intracellular Sf-rhabdovirus RNA production. If some viral genomes had remained, they also could be eliminated by further single-cell cloning.

Protocol #4

The cells from protocol #2 that had been adapted once to grow at 34° C. were cloned in Grace's insect media (Life Technologies/Invitrogen, Inc.) containing DMEM and 10% fetal bovine serum (Hyclone, Inc.). Based on an estimated cloning efficiency, the cells were plated on a 96-well cell culture plate at 10, 30, or 100 cells per well and grown at 28° C.

Five virus-free clones were identified. These clones were expanded onto 24 well plates using serum-free suspension media SF-900 III SFM culture medium (Life technologies/Invitrogen) and then further expanded onto static 6 well plates, static T-75 flasks, and then into shaking (135 rpm) 250 ml Erlenmeyer flasks.

All of these five clones were tested for viral load and other properties. None of the clones contained detectable intracellular Sf-rhabdovirus RNA by RT-qPCR. All of the clones adapted to grow in shaker flasks in serum-free media. Doubling times for three of the five clones ranged from 26.7 hours to 38 hours, comparable to the 26.0 hour doubling time of the parental cells infected with rhabdovirus. Cells of two of the five clones were slightly aggregated, while three clones maintained a single cell state. Clones were selected for the fastest doubling time, for full adaptation to growth in shaker flasks in serum-free media, and for growth as a non-aggregated, single cell suspension.

Virus-free clones obtained by the foregoing methods are selected for further analysis. An example selected clone grew at a normal rate, had comparable viability after freezing, and yielded rAAV after baculovirus infection at amounts that were about 15% higher than the parental cells infected with rhabdovirus.

Thus, the methods of this example generated a population of Sf9 cells that do not produce intracellular or extracellular Sf-rhabdovirus nucleic acids and exhibit 15% higher productivity for producing rAAV than the parental cells.

Follow-up characterization of progeny of the clones produced by the foregoing methods showed that the cells remained virus-free after multiple passages, including after expansion for 8 passages and growth in a 5 L bioreactor with a 3 L working volume. The cells remained virus free (no detectable intracellular or extracellular Sf rhabdovirus by RT-qPCR) after 24 passages, over a time period of four months. Clones were infected with baculovirus for rAAV production and cultured in shaker flasks. The supernatant was harvested several days post infection. The specific productivity (Qp) was then assessed using ddPCR and calculated by dividing the final titer (vector genomes (vg)/ml of harvest fluid) by peak viable cell density (number of cells/ml). The top three producers had Qp's ranging from 2.35×10⁵ to 3.58×10⁵ vg/cell.

### Example 2

### Curing Sf9 Insect Cells of Sf-Rhabdovirus Infection with Antiviral Agents

Sf9 cells are cured of adventitious virus by culturing the infected cells with an antiviral agent. Sf9 cells (8×10⁴ per well) are plated on a 96-well cell culture plate in 200 microliter(s) (μl) Grace's insect media (Invitrogen, Inc.) containing 10% fetal bovine serum (Hyclone, Inc.). In addition, 200 μl Grace's insect media containing 10% fetal bovine serum alone is added to one well to allow measurement of background in a cell viability assay (see below). The cells are incubated for 24 hours at 27° C., 0% CO2 to allow cells to attach to plate.

Antiviral agents, such as ribavirin, individually or in combination at desired dilutions, or vehicle alone as control, is added to the wells. If a siRNA is used to inhibit infection, it is introduced into the cells using highly efficient transfection reagents at a siRNA concentration dependent on the chemical nature of the siRNA used. A FITC-conjugated, non-specific siRNA is also transfected as a control to measure transfection efficiency. The maximum amount of inhibition that can occur is dependent on the transfection efficiency. For example, even if a siRNA is 100% effective, if it is only transfected into 1% of cells, then the maximum RNA knockdown that can occur is 1%. Therefore, ideally 100% of cells would be transfected with a siRNA.

The media (containing the antiviral agent) is changed every 24 hours to remove as much infectious Sf-rhabdovirus as possible and to maximize the antiviral activity of agent(s). The media is harvested once per week, or as desired, to determine the genomic and infectious titers of Sf-rhabdovirus until a statistically significant reduction in viral production is observed compared to an untreated control. When the cells become confluent, the cells are passaged to a new 96-well plate.

To rule out the possibility that the reduction in apparent viral titer is due to cytotoxicity of the antiviral agent or vehicle, the viable cell number is measured by adding 20 μl CellTiter-Blue reagent (Promega, Inc.) to cells in each well at the same time titers are determined. Also 20 μl CellTiter-Blue reagent is added to 200 μl Grace's insect media containing 10% fetal bovine serum (no cells) in order to measure assay background. The cells are incubated 1 hour at 37° C. and fluorescence is measured (e.g., using Molecular Devices SpectraMax2e plate reader) according to the CellTiter-Blue reagent instructions. The background fluorescence due to the reagent alone is subtracted from the fluorescence produced by the cells, then % viability of treated cells relative to the untreated cells is calculated.

### Cured Cells and Infectious Titer Reduction

The cells are considered cured from adventitious virus when the virus is not detected for at least 5 passages beyond the point the cells would be used for protein or rAAV production. For example, if one starts with 1 ml of frozen cells and expands them to a 100 L production culture that represents a 100,000 fold expansion of cells. In each passage, cell number is expanded about 10-fold, so 5 passages would be required for the entire production process and, therefore, no adventitious virus should be detected for 10 passages beyond the point they are initially believed to be cured. For rAAV production, there are a number of steps before cell expansion for production. For example, master and working cells banks of cells are normally generated and typically the working cell bank form (e.g. 1 ml of frozen cells) is thawed to begin rAAV production. After the adventitious agents are removed from the cells, a master cell bank is prepared. A working cell bank is then made from the master cell bank, which entails passaging the cells. As such it can be estimated that at least 20 passages might be needed to demonstrate curing adequate to be used for rAAV production.

Alternatively, adventitious virus might be declared to be cleared from a purification process when independent clearing steps result in at least a 6 log reduction in titer below the titer of adventitious virus in a human dose of protein (or rAAV) if it had not been cleared. For example, the titer of rhabdovirus is typically 10-fold higher than the cell number which would typically be 10⁶ cells/ml. Therefore, if a 100 L culture of cells is required to produce one dose of rAAV, that culture would contain about 10¹¹ cells total and 10¹² Sf-rhabdoviruses total. For example in this case, an 18 log reduction of virus is indicative of a protein or rAAV production being adventitious agent-free. This could, for example, be achieved by incorporating 3 clearing steps, each of which results in a 6-log reduction in adventitious virus titer. See, The European Agency for the Evaluation of Medicinal Products: Human Medicines Evaluation Unit. Committee for Proprietary Medicinal Products (CPMP). Note for Guidance on Virus Validation Studies: The Design, Contribution and Interpretation of Studies Validating the Inactivation and Removal of Viruses (CPMP/BWP/268/95), revised 14 Feb. 1996.

Ideally in removing adventitious agents from the cells, there would be a reduction of both genomic and infectious titer with no reduction of cell viability. A reduction of infectious titer is most preferred and could occur without a reduction of genomic titer (for example if “defective viruses” that package partial genomes are produced). If there is a reduction of both viral titer and cell viability, then it is likely that the antiviral agent is killing cells which in turn results in lower virus production. If that is the case, then it may be necessary to lower the concentration of the antiviral agent. If there is no reduction of viral titer then the antiviral agent is not effective. Some compounds may increase viral production and they would not be useful.

### Testing Cured Cells

Once cells are thought to be cured of Sf-rhabdovirus infection to the level of sensitivity possible by the qPCR based genomic and infectious titer methods, it is desirable to characterize the cells by other methods that can detect Sf-rhabdovirus such as next generation (“deep”) sequencing of cellular RNAs (transcriptome profiling) or transmission electron microscopy of cells or concentrated cell culture media. In the method of next generation sequencing, total RNA is prepared from cells suspected of being cured of viral infection. The RNA is sequenced to a degree such that every RNA in the cells is sequenced multiple times. Hence the sensitivity is such that even very low copy numbers of RNAs in a large number of cells (e.g., 1 RNA per billion cells) can be detected. In Sf9 cells approximately 1 RNA per 1000 RNAs sequenced was a Sf-rhabdovirus RNA. Hence deep sequencing can demonstrate at least a 6 log reduction in the amount of RNA present in the cell. After a variety of methods are used to demonstrate Sf9 cells have been cleared of Sf-rhabdovirus infection the cells are tested for baculovirus-mediated rAAV production since it is possible one or more Sf-rhabdovirus genes (e.g., the N protein that may be functionally equivalent to the adenovirus E2A protein) may serve as helpers to enable rAAV production.

### Example 3

### Determining the Genomic Titer of Sf-Rhabdovirus

Sf-rhabdovirus is a single stranded (negative-strand) RNA virus. Therefore, in order to determine the number of viral RNAs in a sample (genomic titer), the viral RNA is isolated, reverse transcribed, then subjected to qPCR. This is an example of an assay for determining the genomic titer that may be carried out with any of the methods of the invention.

Collect up to 140 μl of sample to be analyzed which can be, for example, infected cells, media from infected cells or fractions from any step in a biologic purification process such as eluates from chromatography columns used to process products produced from infected cells. For samples of cell culture media, the cells are removed from the collected media by centrifugation at 20033 g for 5 min at 20° C. To remove all cells, the media can be passed through a 0.45 micron (μm) filter.

Viral RNA from sample is prepared using the QIAamp viral RNA mini kit (Qiagen, Inc.) according to the manufacturer's instructions. The viral RNA is eluted from the QIAamp column one time in a 60 μl volume and the viral RNA is reverse transcribed using the iScript RT kit (Bio-Rad, Inc.) according to the manufacturer's instructions except that a Sf rhabdovirus M gene-specific primer (Sf-R-forward: 3755-3775; 5′-TCAGGGCAATTCTTCACTCTC-3′; SEQ ID NO: 1) was also added to produce viral cDNA.

Reverse transcriptase (RT) reactions are performed in a total volume of 20 μl it using the following conditions: 4 μl 5× iScript reaction mix (BioRad, Inc.), 1 μl iScript reverse transcriptase (BioRad, Inc.), 1 μl 100 micromolar (μM) Sf rhabdovirus M gene-specific primer (Sf-R-forward: 3755-3775; 5′-TCAGGGCAATTCTTCACTCTC-3′; SEQ ID NO: 1), and 14 μl of purified viral RNA. The reaction was incubated for 30 min at 42° C. and then subjected to a qPCR reaction to quantitate the amount of Sf-rhabdovirus M gene produced in the RT reaction.

The resulting viral cDNA is subjected to qPCR using a forward primer (Sf-R-forward: 3755-3775; 5′-TCAGGGCAATTCTTCACTCTC-3′; SEQ ID NO: 1), a reverse primer (Sf-R-reverse: 3862-3841; 5′-GTTGTTGCTGATCCCATTCATC-3′, SEQ ID NO: 2), a probe (Sf-R-probe: 3794-3817; 5′-6FAM-CTCCAACCGCTCCTTCTCCTCCAC-TAMRA-3′; SEQ ID NO: 3) and 2× TaqMan Fast Advanced Master Mix (Life Technologies). A synthetic DNA oligonucleotide corresponding to the cDNA sequence of the region that is amplified in the qPCR was used as a quantification standard. The sequence of that standard (named Sf-R-template) is: 3755-3817; 5′-TCAGGGCAATTCTTCAC TCTCAAAGTCGTGACATGTGGTCTCCAACCGCTCCTTCTCCTCCACCCACATATGAA GATGGATCCTCAGATGAATGGGATCAGCAACAAC-3′; SQ ID NO: 4). The primers amplify a region of the Sf-rhabdovirus matrix (M) gene that is 108 base pairs long. The probe is a standard TAQMAN® -type probe. The primers, probe and standard are obtained from Sigma, Inc. Sf-R-forward: 3755-3775 and Sf-R-reverse: 3862-3841 primers are desalted without additional purification. Sf-R-probe: 3794-3817 is purified by HPLC. qPCR is preformed using the following conditions and Ct values were converted to titers, expressed in units of vg/ml:
i. Pre-incubation: 50° C., 2 min (for Uracil-DNA glycosylase (UNG) activation)
ii. Pre-incubation #2: 95° C., 20 sec (for Taq polymerase activation)
iii. Incubation #1: 95° C., 3 sec (for DNA denaturation)
iv. Incubation #2: 60° C., 30 sec (for DNA annealing/extension)
   (Repeat steps iii. and iv. for 60 cycles)
v. Incubation #3: 4° C., forever (to store completed reactions)

### Results

qPCR amplification of the standard (Sf-R-template: 3755-3817) using Sf-R-forward primer 3755-3775 and Sf-R-reverse primer 3862-3841 was carried out. The amplified standard was detected with Sf-R-probe 3794-3817 plotted to form a linear curve. In the exemplary assay, the PCR efficiency was 95.5% and the lower limit of sensitivity was 2.2×10⁻⁷ copies in this example.

Specifically, the Sf-rhabdovirus viral genomic titer was 7.2×10⁵ vg per ml in this example. No signal was obtained when either viral RNA or reverse transcriptase was omitted from the reverse transcriptase reactions. The lower limit of detection in this example was at least 6.3×10⁻⁵ vg/ml or 1 vg per 15.9 liters of cell culture media (no fluorescence detected after 60 PCR cycles).

### Example 4

### Determining the Infectious Titer of Sf-Rhabdovirus

Determination of a viral infectious titer is used as a measure of functional viruses capable of infecting a cell (whereas a genomic titer measures the presence of the viral genome which, in some cases, may correspond to inactivated virus that is not capable of infecting a cell).

To determine infectious titer, plate 3×10⁴ indicator cells per well in every well of a 96-well plate, and incubate the cells at 27° C., 0% CO₂ for 24 hours. Indicator cells may include High Five cells (Invitrogen, Inc.) grown in Express Five media, 20 mM glutamine (Invitrogen, Inc.) or Grace's insect media (Invitrogen, Inc.) containing 10% fetal bovine serum media (Hyclone, Inc.).

The cells are infected with serially diluted virus using a multichannel pipette. Make the desired dilution of virus in the next row then continue to dilute the virus in uniform increments in the remaining rows with at least one row of wells that are uninfected. The infected cells are incubated at 27° C., 0% CO₂ for 24 hours. The cells are washed twice with 200 μl media and 200 μl media lacking virus is then added. The purpose of this washing step is to remove any input virus so that only virus that has actually infected the cells and resulted in new virus production will be measured using RT-qPCR. The cells are then incubated at 27° C., 0% CO₂ for 48 hours to allow for new virus production.

To identify which dilution of virus resulted in virus production viral RNA is prepared and quantified by RT-qPCR. Collect 100 μl media and prepare viral RNA from every well using a ZR96 viral RNA kit (Zymo Research, Inc.) or a similar kit according to the manufacturer's instructions. The viral RNA is converted to cDNA using a reverse transcriptase reaction using the Sf rhabdovirus M gene-specific primer (Sf-R-forward: 3755-3775) and incubating for 30 minutes at 42° C. as above. For control reactions, omit viral RNA or reverse transcriptase.

The amount of cDNA produced by PCR is then quantitated using the forward primer (Sf-R-forward: 3755-3775; SEQ ID NO: 1) and reverse primer (Sf-R-reverse: 3862-3841; SEQ ID NO: 2) and probe (Sf-R-probe: 3794-3817; SEQ ID NO: 3). A synthetic DNA oligonucleotide corresponding to the cDNA sequence of the region that is amplified in the qPCR was used as a quantification standard. The sequence of that standard (named Sf-R-template: 3755-3817) is: 5′-TCAGGGCAATTCTTCACTCTCA AAGTCGTGACATGTGGTCTCCAACCGCTCCTTCTCCTCCACCCACATATGAAGATGG ATCCTCAGATGAATGGGATCAGCAACAAC-3′ (SEQ ID NO: 4)

The primers amplify a region of the Sf-rhabdovirus matrix (M) gene that is 108 base pairs long. The probe is a standard TAQMAN®-type probe. The primers, probe and standard are obtained from Sigma, Inc. Sf-R-forward: 3755-3775 and Sf-R-reverse: 3862-3841 were desalted without additional purification. Sf-R-probe: 3794-3817 was purified by HPLC.

qPCRs are performed on a Roche LightCycler 480 II machine. The PCR program used was:
i. Pre-incubation: 50° C., 2 min (for UNG activation)
ii. Pre-incubation #2: 95° C., 20 sec (for Taq polymerase activation)
iii. Incubation #1: 95° C., 3 sec (for DNA denaturation)
iv. Incubation #2: 60° C., 30 sec (for DNA annealing/extension)
v. Repeat steps iii. and iv. for 60 cycles
vi. Incubation #3: 4° C., forever (to store completed reactions)

Ct values from the Q-PCRs were converted to titers, expressed in units of vg/ml, using a Microsoft Excel spreadsheet. All titers over 1 vg/ml are considered positive, which represents cells that were infected and produced virus. The infectious titer (TCID50/ml) is calculated using the Spearman and Karber algorithms.

### Results

qPCR amplification of the standard (Sf-R-template: 3755-3817) using Sf-R-forward: 3755-3775 and Sf-R-reverse: 3862-3841 was carried out. The amplified standard detected with Sf-R-probe: 3794-3817 was plotted to form a linear curve. In the exemplary assay, the lower limit of detection was 1×10⁻³ copies in the sample.

Representative results of a RT-qPCR data analysis to determine the genomic titer of Sf-rhabdovirus produced by infected Sf9 cells are provided in Table 1.

TABLE 1

| | | | | | | | | |
|---|---|---|---|---|---|---|---|---|
| Sample | 100 μl | 10 μl | 1 μl | 1e-1 μl | 1e-2 μl | 1e-3 μl | 1e-4 μl | No |
| name: | Sf-rhabdovirus | Sf-rhabdovirus | Sf-rhabdovirus | Sf-rhabdovirus | Sf-rhabdovirus | Sf-rhabdovirus | Sf-rhabdovirus | Sf-rhabdovirus |
| vg/ml | 92,000 | 34,000 | 6,700 | 1,100 | 0.00004 | 0.00004 | 0.00004 | 0.00004 |
| sample: | | | | | | | | |

The genomic titers of virus produced in positive wells where cells were infected ranged from 1.1×10³ to 9.2×10⁴ vg/ml in this example and are seven logs above that of uninfected cells. Cells infected with 0.1 μl Sf-Rhabdovirus replicated that virus while cells infected with 0.01 μl Sf-Rhabdovirus did not replicate any virus, so there was at least 1 infectious virus in 0.1 μl Sf-Rhabdovirus, but no infectious virus in 0.01 μl Sf-Rhabdovirus. The genomic titers of replicated Sf-rhabdovirus in negative (uninfected) wells was, at most, 3.6×10⁻⁵ vg/ml in this example, which was the lower limit of detection. Hence it is very straight forward to score infected and infected cells in order to obtain the data necessary to calculate an infectious titer (TCID50) by algorithms, such as the commonly used Spearman and Karber algorithm. Uninfected cells did not produce Sf-Rhabdovirus and that is a necessary negative control and condition to obtain infectious titer data. That is, the cells used for the assay must not be infected with the virus that is to be detected.

The Spearman and Karber algorithms were used to calculate infectious titer (TCID50/ml). The infectious titer of virus was 3.1×10⁴ vg/ml in this example. The genomic titer of undiluted virus (determined in a separate experiment) was 3.9×10⁵ vg/ml in this example. Therefore, the ratio of genomic titer to infectious titer was 12.6 in the above examples. The ratio of genomic titer to infectious titer for many viruses is in a range of 10-1000 so the ratio of genomic titer to infectious titer determined for Sf-rhabdovirus in this example is within a typical range.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed:

1. A method of removing adventitious virus from cells comprising:
(a) growing cells in cell culture at a suboptimal temperature that is higher or lower than (i) a first optimal temperature range for replication of the adventitious virus or (ii) a second optimal temperature range for replication of the cells, for at least 2 passages, and
(b) detecting a reduction of the adventitious virus in the cell culture following growth of the cells at the temperature.

2. The method of claim 1 further comprising the step of isolating a cell from the cell culture and separately culturing the cell.

3. The method of claim 1 wherein the suboptimal temperature is at least 3° C. higher than the first or second optimal temperature range.

4. The method of claim 1 wherein the suboptimal temperature is at least 3° C. lower than the first or second optimal temperature range.

5. The method of claim 1 wherein the suboptimal temperature is at least 6° C. higher or lower than the first or second optimal temperature range.

6. The method of claim 1 wherein the suboptimal temperature is at least 6° C. lower than the first or second optimal temperature range.

7. The method of claim 1 further comprising, prior to the growing step, adapting the cells to grow at the suboptimal temperature by gradually changing the temperature of the cell culture over a time period to the suboptimal temperature.

8. The method of claim 7 wherein the temperature of the cell culture is changed by at least 0.1° C. after each passage.

9. The method of claim 7 wherein the temperature of the cell culture is changed by at least 1° C. after each passage.

10. The method of claim 7 wherein the time period is a time in which the cells are passaged at least for 4 times.

11. A method of removing adventitious virus from cells comprising:
(a) growing cells in cell culture for at least five passages in the presence of one or more antiviral agents in an amount effective to remove or cure the adventitious virus from the cell culture, and
(b) detecting a reduction of the adventitious virus in the culture following growth in the presence of the one or more antiviral agents.

12. The method of claim 11 further comprising the step isolating a cell from the cell culture and separately culturing the cell.

13. The method of any of claims 1-12 wherein the cell is an insect cell or a mammalian cell.

14. The method of any one of claims 1-12, wherein the cell is from Spodoptera frugiperda cells, Trichoplusia ni cells, Ascalapha odorata cells, or Aedes albopictus cells.

15. The method according to claim 13 or 14, wherein the insect cell is a Sf9 cell.

16. The method of any one of claims 1-15 wherein the adventitious virus is Mononegavirales, Rhabdovirus, sf-Rhabdovirus, Cytohabdovirus, Ephemeovirus, Nodaviruis, Norvihabdovirus, Nucleorhabdovirus, Perhabdovirus, Sigmavirus, Sinistar-like virus, Sprivivirus, Tibrovirus, Tupavirus, Vesiculorirus, Taastrup virus, Nakha virus, Chandipura virus, Lettuce yellow mottle virus, Ivy vein banding virus isolate, or Durham virus.

17. The method of any one of claims 1-15 wherein the adventitious virus is Mononegavirales virus.

18. The method of any one of claims 1-15 wherein the adventitious virus is Rhabdovirus.

19. The method of any one of claims 1-15 wherein the adventitious virus is Sf-Rhabdovirus.

20. The method of any one of claims 11-19, further comprising the steps of quantitatively measuring the adventitious virus before growth in the presence of the antiviral agent.

21. The method of any one of claims 1-20, wherein the adventitious virus is detected by measuring genomic titer of the virus or by determining infectious titer of the virus.

22. The method of any one of claims 11-21 wherein the one or more antiviral agents inhibits activity of one or more of a nucleocapsid, phosphoprotein, matrix, envelope, RNA-dependent RNA polymerase, X protein, N protein, P protein, M protein, G protein, and L protein.

23. The method of any one of claims 11-21 wherein the antiviral agent includes one or more of an siRNA, antisense RNA, phosphonoformate, phosphonoacetate, ara-ATP, ribavirin, sinefungin, selenomethionine, chloroquine, Argonaute up-regulators, Dicer up-regulators, Ars2 up-regulators, Metformin, saquinavor, nelfinavir, cystatin A, MG132, bortezomib, leptomycin B, azodicarbonamide, and sodium selenite.

24. The method of any one of claims 11-21 wherein the antiviral agent inhibits expression of a viral gene product related to viral replication.

25. The method of claim 24 wherein the viral gene product is encoded by one or more of an X gene, N gene, P gene, M gene, G gene, and L gene.

26. A cell produced by or obtainable by the method of any of claims 1-25 that has no detectable virus intracellularly or extracellularly.

27. The cell of claim 26 where the cell is an insect cell.

28. The cell of claim 26 wherein the insect cell is a Spodoptera frupperda cell and the virus is Rhabdovirus.

29. The cell of claim 26 wherein the insect cell is Trichoplusia ni and the virus is Nodavirus.

30. The cell of claim 26 wherein the insect cell is a High Five cell and the virus is Nodavirus.

31. The cell of any of claims 26-30 grown in culture that is at least 5 L in volume.

32. The method of claim 1 further comprising, prior to the detecting step, growing the cells in the presence of one or more antiviral agents in an amount effective to reduce or eliminate the adventitious virus from the cell culture.

33. The method of claim 1 wherein the growing step includes adding one or more antiviral agents in an amount effective to reduce or eliminate the adventitious virus from the cell culture.


SEQUENCE LISTING (4 sequences)

SEQ ID NO: 1 - DNA, 21 bp, Artificial Sequence
  Forward primer
tcagggcaattcttcactctc

SEQ ID NO: 2 - DNA, 22 bp, Artificial Sequence
  Reverse primer
gttgttgctgatcccattcatc

SEQ ID NO: 3 - DNA, 24 bp, Artificial Sequence
  Probe sequence
  misc_feature (1)..(1): 5′ 6-FAM
  misc_feature (24)..(24): 3′-TAMRA
ctccaaccgctccttctcctccac

SEQ ID NO: 4 - DNA, 108 bp, Artificial Sequence
  Template sequence
tcagggcaattcttcactctcaaagtcgtgacatgtggtctccaaccgctccttctcctccacccacatatgaagatggatcctcagatgaatgggatcagcaacaac